(19) United States
(12) Patent Application Publication
(10) Pub. No.: US 2026/0086058 A1
(43) Pub. Date: Mar. 26, 2026

(54) IMPEDANCE-BASED ASSESSMENT OF BLOOD SAMPLES

(71) Applicants: Case Western Reserve University, Cleveland, OH (US); Emory University, Atlanta, GA (US)

(72) Inventors: Pedram Mohseni, Highland Heights, OH (US); Umut A. Gurkan, Shaker Heights, OH (US); Michael A. Suster, Cleveland Heights, OH (US); Vivien A. Sheehan, Atlanta, GA (US); Solomon Oshabaheebwa, Cleveland, OH (US); Christopher A. Delianides, Cleveland, OH (US); Akshay A. Patwardhan, Atlanta, GA (US)

(21) Appl. No.: 19/337,302

(22) Filed: Sep. 23, 2025

Related U.S. Application Data
(60) Provisional application No. 63/698,330, filed on Sep. 24, 2024.

(51) Int. Cl. G01N 27/02; G01N 33/49

(52) U.S. Cl. CPC G01N 27/028; G01N 33/49

(57) ABSTRACT

A described example relates to a system that includes a microfluidic device and an impedance analyzer. The microfluidic device includes a microchannel extending through a portion of a housing. The microchannel is configured to receive a fluid sample including blood cells that flows along in a direction of fluid flow through the microchannel. The microchannel includes a plurality of micropillar arrays along the direction of fluid flow, and each of the plurality of micropillar arrays is located between a respective pair of electrodes in the microchannel. The impedance analyzer can be coupled to each of the electrodes and configured to measure electrical impedance of at least some of the micropillar arrays at multiple times, in a wash-free assay, including at least one impedance measurement before the fluid sample is flowing through the microchannel and at least one impedance measurement after the fluid sample is flowing through the microchannel.

### RELATED APPLICATION

This application claims priority from U.S. Provisional Application No. 63/698,330, filed Sep. 24, 2024, the subject matter of which is incorporated herein by reference in its entirety.

### GOVERNMENT FUNDING

This invention was made with government support under AI176469, EB027690, HL162214, and HL165946 awarded by the National Institutes of Health. The government has certain rights in the invention.

### TECHNICAL FIELD

This description relates to assessing properties of biological fluids and, more particularly, to impedance-based measurements to assess red blood cell deformability.

### BACKGROUND

Red blood cell (RBC) deformability, or flexibility, enables proper blood flow in microvessels. In RBC disorders, such as sickle cell disease (SCD), poorly deformable RBCs may fail to squeeze through narrow microcapillary openings, causing vaso-occlusion and ischemia that can lead to organ damage and pain events. While therapy strategies exist to increase the proportion of functional hemoglobin, the primary endpoint of many such strategies is a reduction in pain. However, since individuals with SCD or other disorders may have few or no pain events on non-curative therapies, such as chronic transfusion, reduction in pain events alone is not evidence of the normalization of RBC function.

Tools have been developed to assess deformability, such as the laser-assisted optical rotational red cell analyzer, which is based on ektacytometry. This instrument deforms cells through either mechanical shear or an osmolarity gradient, measures the deformation of cells through laser diffraction, and continuously adjusts the oxygen tension to observe changes in deformability under increasing levels of hypoxia. Ektacytometry is a difficult technique for clinical translation due to instrument size, technical complexity, and the cost associated with its mechanical and optical parts. Also, ektacytometry and other existing techniques measure RBC deformability in a static environment and do not replicate the flow environment in which RBCs naturally deform in vivo, limiting the physiologic relevance of these measurements.

Microfluidic-based assays have also been developed for RBC deformability assessment. Common approaches include elongation-based systems that measure cell shape changes under applied shear and transit-based systems that quantify the transit time or pressure needed for RBCs to pass through constrictions. These existing systems, however, suffer from limitations such as low throughput due to clogging of narrow channels by stiff RBCs and laborious image-processing steps needed to extract individual cell-deformation data. The low throughput leads to reduced assay sensitivity, since most diseases alter the deformability of only a small subpopulation of RBCs, which necessitates the analysis of a large number of cells to obtain clinically meaningful results. Additionally, these systems require high-speed imaging devices that increase the cost and complexity of the assay.

### SUMMARY

This description relates to assessing properties of biological fluids and, more particularly, to impedance-based measurements to assess red blood cell deformability.

In one example, a system for wash-free electrical impedance-based assessment of blood cell properties is described. The system includes a microfluidic device and an impedance analyzer. The microfluidic device includes a microchannel extending through a portion of a housing. The microchannel is configured to receive a fluid sample including blood cells that flows along in a direction of fluid flow through the microchannel. The microchannel includes a plurality of micropillar arrays along the direction of fluid flow, and each of the plurality of micropillar arrays is located between a respective pair of electrodes in the microchannel. The impedance analyzer can be coupled to each of the electrodes and configured to measure electrical impedance of at least some of the micropillar arrays at multiple times, in a wash-free assay, including at least one impedance measurement before the fluid sample is flowing through the microchannel and at least one impedance measurement after the fluid sample is flowing through the microchannel.

In another example, a method includes perfusing a fluid sample including red blood cells into at least one microchannel of a microfluidic device. The at least one microchannel can include a plurality of micropillar arrays arranged in a direction of fluid flow through the at least one microchannel, and each of the plurality of micropillar arrays can be located between a respective electrode pair. The method also includes measuring an electrical impedance of at least one micropillar array between a respective electrode pair including at least one measurement before the fluid sample has entered the microchannel and at least one measurement after the fluid sample has entered the microchannel. The perfusion of the fluid sample and the measuring of electrical impedance can be performed in the absence of washing of the microchannel. The method also includes determining a normalized impedance change for the at least one micropillar array based on the measurements of the electrical impedance. The method also includes determining an assessment of deformability and/or microcapillary occlusion of the red blood cells in the fluid sample based on the normalized impedance change determined for the at least one micropillar array.

In yet another example, this description provides a method of measuring efficacy of a therapeutic agent in modulating red blood cell adhesion and/or deformability. The method includes perfusing a fluid sample including red blood cells through at least one microchannel of a microfluidic device. The at least one microchannel can include a plurality of micropillar arrays arranged in a direction of fluid flow through the at least one microchannel, and each of the plurality of micropillar arrays can be located between a respective pair of electrodes. The method also includes measuring an electrical impedance of at least one micropillar array in the at least one microchannel before and after perfusion of the fluid sample through the at least one microchannel. The perfusion of the fluid sample and the measuring of the electrical impedance can be performed wash-free. The method also includes determining a normalized impedance change for the at least one micropillar array based on the electrical impedance measured before and after the fluid sample is being perfused through the microchannel. The method also includes determining the measure of efficacy of the therapeutic agent based on a comparison of the normalized impedance change for the at least one micropillar array to a control.

### BRIEF DESCRIPTION OF THE DRAWINGS

FIG. 1 is a block diagram depicting an example system for electrical impedance-based assessment of blood cell deformability.

FIG. 2 depicts another example system for electrical impedance-based assessment of blood cell deformability.

FIG. 3 is a graph depicting a comparison of impedance measured by an example miniaturized impedance analyzer and a benchtop impedance analyzer.

FIG. 4 is a graph depicting impedance changes over time for a plurality of multiple micropillar arrays.

FIG. 5 is a schematic diagram showing an example of contributions of free and occluding RBC samples within multiple micropillar arrays.

FIG. 6 is a graph depicting examples of occlusion indexes of multiple RBC samples observed before and after a wash step.

FIG. 7 depicts images of micropillar arrays at different times during sample perfusion.

FIG. 8 is a graph depicting examples of impedance-based occlusion indexes determined for a wash-free assay correlated with optical occlusion indexes for multiple blood cell samples.

FIG. 9 is a graph depicting examples of impedance-based occlusion indexes determined for a post-wash assay correlated with optical occlusion indexes for multiple blood cell samples.

FIGS. 10A and 10B are bar graphs depicting impedance-based occlusion indexes determined for a wash-free assay for multiple blood cell samples, measured using devices at two different locations operated by two different trained users.

FIGS. 11A and 11B are graphs depicting variations in impedance-based occlusion indexes across users and locations for a wash-free assay implemented in accordance with an example embodiment of the invention.

FIGS. 12A-12E are graphs demonstrating correlations between the wash-free occlusion index and biomarkers of RBC hemolysis.

FIGS. 13A-13F are graphs demonstrating experimental results and correlations based on measurements made using a prototype MIRCA assay.

FIG. 14 is a flow diagram illustrating an example method for electrical impedance-based assessment of a fluid sample.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also include the general class of which a specific example may be used for illustration. The terminology herein is used to describe specific aspects of the systems and methods described herein, but their usage does not delimit the invention, except as set forth in the claims.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2%, or 1% to a reference quantity, level, value, number, frequency, percentage, dimension, size, amount, weight, or length. In one example, the term “about” or “approximately” refers to a range of quantity, level, value, number, frequency, percentage, dimension, size, amount, weight or length±15%, ±10%, ±9%, ±8%, +7%, ±6%, ±5%, ±4%, ±3%, ±2%, or ±1% about a reference quantity, level, value, number, frequency, percentage, dimension, size, amount, weight, or length.

The term “microchannels” as used herein refers to pathways through a medium, e.g., silicon, that allow for movement of liquids and gasses. Microchannels can therefore connect other components, i.e., keep components “in fluid communication.” While it is not intended that the present application be limited by precise dimensions of the channels, illustrative ranges for channels are as follows: the channels can be between 0.1 and 100 μm in depth (e.g., 50 μm) and between 50 and 10,000 μm in width (e.g., 400 μm). The channel length can be between 1 mm and 100 mm (e.g., about 27 mm).

The term “patient” or “subject” as used herein refers to a human or animal and need not be hospitalized. For example, out-patients and individuals in nursing homes are “patients.” A patient may comprise any age of a human or non-human animal and therefore includes both adult and juveniles, i.e., children. It is not intended that the term “patient” connotes a need for medical treatment and, thus, a patient may voluntarily or involuntarily be part of experimentation whether clinical or in support of basic science studies.

The term “sample” as used herein is used in its broadest sense and includes environmental and biological samples. Environmental samples include material from the environment such as soil and water. Biological samples may be animal, including human, fluid, e.g., blood, plasma, and serum; solid, e.g., stool; tissue; liquid foods, e.g., milk; and solid foods, e.g., vegetables. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

Example embodiments described herein relate to systems, devices, and methods to perform electrical impedance measurements for assessment of red blood cell (RBC)—mediated microvascular occlusion associated with abnormal RBC deformability. For example, a system includes a microfluidic device that includes one or more microchannels having impedance elements (e.g., micropillar arrays) arranged in a direction of fluid flow through the microchannel. The microfluidic devices described herein can mimic architectural features associated with capillary beds of a subject. For example, a microfluidic device can mimic 20-μm to 4-μm narrow blood vessels to replicate the specific deformation of RBCs when traversing the microvascular bed through capillaries and small venules. Examples of microfluidic devices that can be used to implement the systems and methods described herein are disclosed in PCT International Publication No. WO 2022/120139, which is incorporated herein by reference in its entirety.

Each of the micropillar arrays (or other impedance elements) can be located between a pair of electrodes within the microchannel. For example, impedance measurements can be determined for each of a plurality of micropillar arrays at times both before and after a fluid sample (e.g., including red blood cells) perfuses through the microchannel. An impedance change can be determined based on the impedance measurements for each of the micropillar arrays to provide an impedance-based assessment of the red blood cells in the fluid sample under test. The impedance-based assessment can provide a readout representative of deformability and/or microcapillary occlusion of the red blood cells in the fluid sample. In some examples, an occlusion index (OI) can be calculated based on the impedance change that has been determined for each of the micropillar arrays. The impedance-based assessment and/or index can be provided to a display (or other output device) as a real-time readout of the impedance-based assessment.

Also, in some examples, the systems and methods described herein can be implemented in a wash-free assay. As used herein, the term “wash-free” refers to a design or operational feature that allows for the processing of samples without requiring extensive washing steps. For example, a wash-free assay can be implemented, in whole or in part, in which the perfusion of fluid samples through the microchannel and impedance measurements are performed in a microchannel in the absence of washing of the microchannel. As described, herein, the wash-free assay is advantageous because it can reduce sample loss, decrease processing time, simplify protocols, and/or facilitate integration with other processes. Thus, the systems, devices, and methods described herein that perform a wash-free assay can be ideal for point-of-care testing applications, in contrast to approaches that include traditional washing steps that can be time-consuming as well as may lead to loss of sample and/or reduced sensitivity. Additionally, in some examples, the systems, devices, and methods are referred to as including or implementing a microfluidic impedance-based red cell assay (MIRCA) or a “MIRCA assay.”

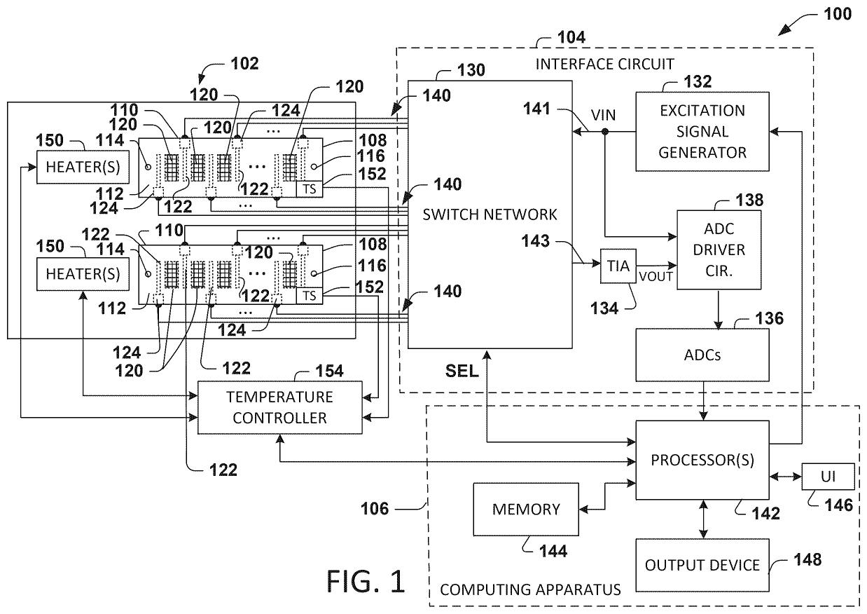

FIG. 1 is a block diagram depicting an example system 100 for electrical impedance-based assessment of one or more samples under test. The following examples describe the system 100 for assessing RBC-mediated microvascular occlusion of a fluid sample that includes red blood cells, which can be associated with various conditions (e.g., malarial infection, long-term blood storage, end-stage renal disease, sickle cell disease (SCD), hereditary spherocytosis, etc.). In other examples, the system 100 can be used to perform electrical impedance-based assessment of other types of samples.

The system 100 includes a sample test platform 102, an interface circuit 104, and a computing apparatus 106. The interface circuit 104 and the computing apparatus 106 can define an impedance analyzer that is configured to measure impedance, perform calculations, and control operation of the system 100 described herein. In some examples, the system 100 and its constituent parts 102, 104, and 106 can be implemented as a portable (e.g., handheld unit), modular device that is readily transportable and readily usable at the point of care. The portable unit is sometimes referred to herein as a miniaturized impedance analyzer (MIA). Also, while the example of FIG. 1 shows the interface circuit 104 and the computing apparatus 106 as separate structures, in other examples, the interface circuit 104 and computing apparatus 106 can be implemented in a single structure (e.g., containing both hardware and software components). Alternatively, one or more parts of the interface circuit 104 and/or the computing apparatus 106 can be distributed to other structures and coupled together through communication interfaces to perform the functions described herein.

The test platform 102 is configured to hold one or more microfluidic devices 108 (two microfluidic devices shown in FIG. 1). In the following example, each of the microfluidic devices 108 is described as being the same or similarly configured. In other examples, the microfluidic devices 108 can be configured differently. Each of the microfluidic devices 108 can be implemented as a chip (e.g., OcclusionChip or another microfluidic structure) that can be connected to the test platform 102. For example, the microfluidic device 108 includes a housing 110 and one or more microchannels 112 in the housing that permits fluid sample flow through an interior of the housing along a length of the microchannel from a first end to a second end of the microchannel. The microfluidic device 108 includes an inlet port 114 and an outlet port 116. The inlet port 114 allows fluid to flow through the microchannel 112 in a direction of fluid flow through the microchannel 112 from the first end to the second end and out the outlet port 116. Although FIG. 1 depicts two microfluidic devices 108, each having a single microchannel 112, in other examples, a given microfluidic device 108 can include more than one microchannel.

In described examples, the fluid sample, which is perfused through one or more of the microchannels 112, can include red blood cells. For example, the fluid sample can include RBCs extracted from whole blood (e.g., by centrifugation). The fluid sample further can include diseased RBCs, such as sickle RBCs. The fluid sample may also include healthy RBCs. In an example, the fluid sample can include whole blood. Also, or alternatively, the sample being perfused through the microchannel 112 can further include one or more other therapeutic agents to modulate blood cell adhesion and/or deformability. Examples of agents can include a mitapivat, a therapeutic agent. The agent can be added directly to the fluid sample that is perfused. Also, or alternatively, a subject can be treated with a therapeutic agent, such as to modulate the adhesion and/or deformability of RBCs, and the fluid sample can include RBCs from the subject's blood already modulated by the therapeutic agent. Also, or alternatively, the fluid sample can be perfused under normoxic or hypoxic conditions. Also, or alternatively, the fluid sample and/or microchannel through which the fluid sample is perfused can be heated (e.g., to a physiologically relevant temperature).

Additionally, an occlusion region of the microchannel 112 includes a plurality of micropillar arrays 120 arranged in the microchannel along the direction of fluid flow. Each of the micropillar arrays 120 can be located between a respective pair of electrodes 122 in the microchannel 112. For example, pairs of uniformly spaced electrodes 122 are positioned on opposite sides of each micropillar array 120 in a direction of fluid flow through the microchannel 112. The micropillar arrays 120 and electrodes 122 can be arranged in series such that a flow path through one micropillar array 120 and over its respective pair of electrodes 122 is in parallel with a flow path through the other micropillar arrays 120 and over other flanking electrodes 122. Additionally, successive micropillar arrays 120 in the microchannel 112 can have progressively decreasing separation distances between the micropillars thereof in the direction of fluid flow. As described herein, the pairs of electrodes 122 can be arranged and configured to measure the electrical impedance of the respective micropillar array 120 that is located between such electrode pair. Each of the electrodes 122 further can be coupled to conductive terminals 124 (e.g., electrically conductive pads), which can be located along opposing longitudinal edges of the microfluidic devices 108.

The interface circuit 104 can be coupled to each of the electrodes 122 through connections to the respective conductive terminals 124 to which the electrodes are connected. In the example of FIG. 1, the interface circuit 104 includes a switch network 130, an excitation signal generator 132, an amplifier 134, an analog-to-digital converter (ADC) 136, and an ADC driver circuit 138. The switch network 130 includes a plurality of input/output (I/O) terminals 140 coupled (e.g., by wires and/or traces) to each of the terminals 124 and the respective electrodes 122 of the microfluidic devices 108. The switch network 130 also has a signal input 141 coupled to an output of the signal generator 132 and a signal output 143 coupled to an input of the amplifier 134. The signal generator 132 can be configured to generate an excitation signal (shown as VIN) that is provided to the signal input of the switch network 130. For example, the excitation signal VIN can be a sinusoidal waveform having a predetermined frequency, such as in a range from about 1 kHz to about 100 kHz (e.g., approximately 10 kHz). The excitation signal VIN can also be provided to an input of the ADC driver circuit 138, which can condition and amplify the excitation signal and provide VIN prior to sampling/conversion by the ADC 136. The ADC can be implemented as a multichannel ADC or include more than one ADC for converting VIN and VOUT to respective digital data.

The switch network 130 is configured (e.g., including multiplexer and demultiplexer circuitry) to route the excitation signal VIN to a selected electrode 122 of a given pair of the electrodes. For example, in response to a selection control signal (SEL), which can be provided by the computing apparatus 106 (or other control circuitry), the switch network 130 routes the excitation signal VIN to a first electrode 122 of a given electrode pair and receives a corresponding response signal from a second electrode 122 of the given electrode pair. The excitation signal VIN can propagate from first electrode 122 through a respective one of the micropillar arrays 120, which is flanked by the given electrode pair, and to the second electrode 122 of the given electrode pair. As described herein, the excitation signal VIN will also propagate through any fluid sample located between the given electrode pair. The switch network 130 is further configured to route a corresponding signal received at the second electrode 122 of the given electrode pair, in response to excitation signal VIN, to the output 143 of the switch network, which is coupled to the input of the amplifier 134.

In an example embodiment, the selection control signal SEL can be provided to operate the switch network 130 to route the excitation signal VIN and the response signal to and from a selected, single pair of the electrodes 122 during each phase of a testing cycle. Various schemes can be used to control successively routing the excitation through each pair of electrodes in the microchannel 112. In examples, such as the system of FIG. 1, which supports two or more microfluidic devices 108, each of the microfluidic devices can be operated concurrently (e.g., by the interface circuit 104 and computing apparatus 106). Separate operation of the microfluidic devices 108 can also be implemented, if desired.

The amplifier 134 is configured to provide an output signal (shown as VOUT) to another input of the ADC driver circuit 138 based on the response signal provided at the signal output 143 of the switch network 130. The amplifier 134 can be implemented as a transimpedance amplifier, which has a feedback resistor ZFB coupled between its output and an inverting input thereof that is coupled to the signal output 143 of the switch network 130. A non-inverting input of the transimpedance amplifier 134 can be coupled to ground. The ADC driver circuit 138 can provide the excitation signal VIN and the output signal VOUT (e.g., conditioned and amplified versions of VIN and VOUT) to the ADC 136. The ADC 136 is configured to convert the excitation signal VIN and the output signal VOUT to digital versions of such signals, which are provided to the computing apparatus 106 for further processing.

In the example of FIG. 1 the computing apparatus 106 includes one or more processors 142, memory 144, a user interface 146, and an output device 148 (e.g., including a display). The computing apparatus 106 can be coupled to the interface circuit 104 and other components of the system 100 through one or more interfaces (e.g., physical connections and/or communication links, which can be wired or wireless). The memory 144 can be implemented as one or more non-transitory machine readable media, which may be resident in the computing apparatus 106 or remotely located from the computing apparatus (e.g., cloud memory). The memory 144 can include data and instructions executable by the processor 142. The processor 142 can be coupled to the memory 144 to access the data and instructions (e.g., program code) stored in the memory. The instructions, when executed by the processor 142, cause the processor to perform respective control logic, functions, and/or methods described herein.

The processor 142 may include any one or more of a microprocessor, a controller, a digital signal processor (DSP), an application-specific integrated circuit (ASIC), a field-programmable gate array (FPGA), or equivalent discrete and/or integrated logic circuitry. In some examples, processor 142 may include multiple components, such as any combination of one or more microprocessors, one or more controllers, one or more DSPs, one or more ASICs, or one or more FPGAs, as well as other discrete or integrated logic circuitry. The term “processor” may generally refer to any of the foregoing logic circuitry, alone or in combination with other logic circuitry, or any other equivalent circuitry. The user interface 146 can receive instructions from a user (e.g., entered via a user input device—not shown) to set operating parameters or define other functions executed by the processor 142.

The processor 142 is configured to determine a measure of electrical impedance based on the excitation signal VIN and the output signal VOUT. As described herein, the excitation signal VIN and the output signal VOUT can be provided to the processor 142 (e.g., by ADC 136) as digital information representing each applied electrical signal (e.g., the excitation signal VIN) and each amplified version of the measured signal (e.g., output signal VOUT) for each phase of each acquisition cycle. The digital representations of the excitation signal VIN and the output signal VOUT can be stored in the memory 144 as electrical data. The electrical data can further include channel information (e.g., specifying each electrode pair and/or micropillar array), timestamps, and other parameters associated with each applied or measured signal that is being monitored.

The system 100, including interface circuit 104 and the computing apparatus 106, with or without the test platform 102, can define an impedance analyzer (e.g., MIA), which is configured to measure the electrical impedance of one or more of the micropillar arrays. For example, the impedance analyzer can acquire one or more first impedance measurements for each micropillar array 120 (or at least some micropillar arrays) before the fluid sample perfuses through the microchannel 112. The first impedance measurements acquired for each micropillar array 120 before the fluid sample is perfusing through the microchannel 112 can define a baseline impedance for the respective micropillar array. The impedance analyzer (e.g., interface circuit 104 and computing apparatus 106) can acquire one or more second impedance measurements for the respective micropillar arrays 120 after the fluid sample is perfusing through the microchannel 112 and the micropillar arrays. The processor 142 further can be configured to determine an impedance change (e.g., a normalized impedance change) based on the first and second impedance measurements for each of the micropillar arrays 120, namely, for measurements obtained both before and after the fluid sample is flowing through the microchannel. The value of the impedance change computed for one or a number of micropillar arrays 120 can provide an assessment of deformability and/or microcapillary occlusion of the red blood cells in the fluid sample.

In some examples, the processor 142 is configured to calculate an index indicative of the assessment of deformability and/or microcapillary occlusion of the red blood cells in the fluid sample based on the impedance change determined for at least some of the micropillar arrays. The index can be computed as an occlusion index (OI) parameter for the red blood cells in the fluid sample, such as described herein (see, e.g., Eqs. 4, 5, and 6).

Also, or alternatively, the processor 142 can be configured to compensate for a contribution of free non-occluding red blood cells and/or other objects present in the microchannel 112. For instance, the processor 142 can be configured to estimate an impedance contribution caused by the free non-occluding red blood cells present in the microchannel, such as based on an impedance measured across a micropillar array 120 (or multiple arrays) having openings sized to allow red blood cells to pass through substantially un-occluded. The impedance can be a normalized impedance change based on impedance measurements before and after perfusion of the fluid sample through the microchannel, such as described herein. The processor 142 can be configured to subtract the estimated impedance contribution from the impedance change determined for each of the micropillar arrays 120 to provide a corresponding compensated impedance change for each of the respective micropillar arrays. The processor 142 further can be configured to calculate an OI parameter for the fluid sample based on the corresponding compensated impedance changes determined for each of the micropillar arrays, such as described herein (see, e.g., Eqs. 5 and 6).

Additionally, as described herein, the processor 142 further can be configured to provide a readout parameter that can be rendered on the output device (e.g., display) 148, such as for analysis by a user (e.g., healthcare provider). The readout parameter can describe one or more of the impedance changes that have been determined for one or more micropillar arrays or one or more values derived from the impedance changes, such as providing an assessment of deformability and/or microcapillary occlusion (e.g., a representation of the OI parameter). In some examples, the output device 148 can include a touchscreen display, which provides the user interface 146 (e.g., a graphical user interface) and operates as a user input device. Also, or alternatively, the computing apparatus 106 can include other types of user input devices (e.g., pointer device, keyboard, keypad, one or more buttons, gesture interface or the like) in other examples.

In some examples, the system 100 can include a heating system configured to heat the one or more microchannels 112 and a fluid sample flowing therethrough. In the example of FIG. 1, the heating system includes one or more heaters 150, one or more temperature sensors 152, and a temperature controller 154. For example, the heaters 150 can be implemented as resistive heaters (e.g., polyimide flexible resistive heaters) that can be attached to a surface (e.g., top and/or bottom surfaces) of the housing of each of the respective microfluidic devices 108. Other types of heaters can be used in other examples, and their placement can likewise vary. The temperature sensors 152 can be implemented as thermistors, thermocouples, or other temperature sensing devices and configured to provide sensor signals to the temperature controller 154 representative of sensed temperature.

The temperature controller 154 can control the heaters 150 to regulate the temperature of the microfluidic devices 108 based on the sensor signals. The temperature controller 154 thus can implement closed-loop control to detect and maintain each of the microfluidic devices 108 at a predetermined temperature. For example, the predetermined temperature can be about 37° C. or another physiologically relevant temperature. The processor 142 can provide a command to the temperature controller 154 to establish and maintain the predetermined temperature, which can be set in response to a user input instruction entered through the user interface 146. While the temperature controller 154 is depicted as separate from the computing apparatus 106, in other examples, the controller may be implemented as part of the computing apparatus (e.g., by a processor and/or instructions programmed to perform the temperature control function). Advantageously, the elevated temperature caused by the heaters 150 can significantly increase the occlusion index in SCD samples consistent with the level of occlusion that occurs at physiological temperatures (e.g., in vivo).

Also, or alternatively, the system 100 can include a flow control system (not shown) that includes one or more pumps (e.g., piezoelectric pumps) that are in fluid communication (e.g., through conduits) with the at least one microchannel 112 of the microfluidic device 108. An example of a flow control system and other features that can be integrated into the system 100 are disclosed in the above-incorporated PCT International Publication No. WO 2022/120139. For example, the flow control system can include a fluid sample reservoir fluidically connected with the one or more microchannels 112, and a pump that perfuses fluid from the fluid sample reservoir to the inlet port 114, through the one or more microchannels 112, to the outlet port 116, and from the outlet port 116 to a waste or fluid collection reservoir. The fluid sample reservoir can include a fluid sample that includes blood cells (e.g., RBCs). The flow control system can control the flow of the fluid sample through one or more microchannels, such as at a physiologically relevant flow velocity (e.g., an average fluid velocity ranging from about 100 μm/s to about 2 mm/s). Also, or alternatively, the pump can be designed and configured to create and regulate a pressure (gauge pressure) in at least one of the microchannels 112. The flow control system can regulate the flow rate and/or pressure based on a sensed fluid flow rate and/or pressure.

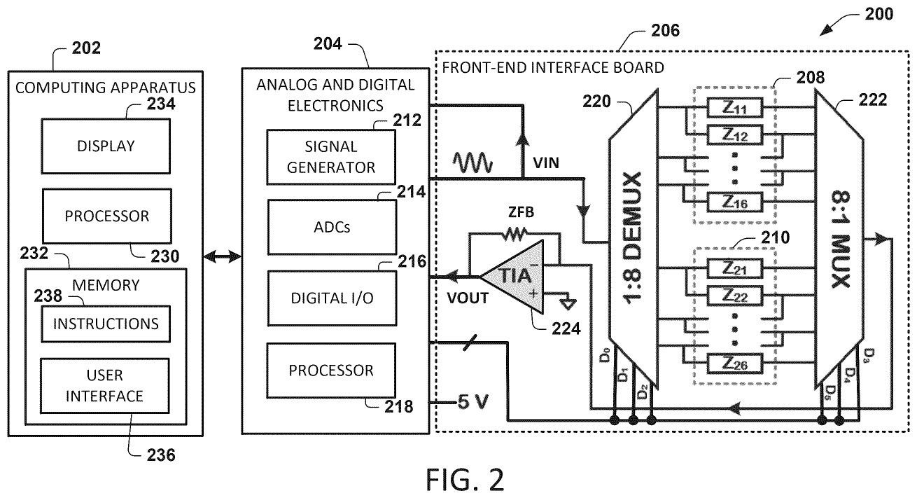

FIG. 2 depicts another example system 200 for electrical impedance-based assessment of blood cell pathology. The system 200 provides an example embodiment that can be used to implement the system 100 of FIG. 1. Accordingly, the description of FIG. 2 may refer to certain aspects of the system 100. The system 200 includes a computing apparatus 202, analog and digital electronics 204, and front-end interface circuitry (e.g., a board) 206, which can define an impedance analyzer.

The front-end interface circuitry 206 includes or is configured to hold one or more microfluidic devices 208 and 210. For example, the microfluidic devices 208 and 210 can be MIRCA microfluidic chips or the OcclusionChip available from BioChip LABS of Cleveland, Ohio. Other microfluidic devices can be used in other examples.

By way of example, each of the microfluidic devices 208 and 210 includes a number (e.g., six) discrete micropillar arrays with openings that narrow along the direction of fluid flow, such as ranging from 12 μm at the inlet port to 3 μm at the outlet port. As one example, the sizes of openings (e.g., separation between micropillars) for each of the micropillar arrays arranged in series in each microfluidic device 208 and 210 can include 12 μm, 10 μm, 8 μm, 6 μm, 4 μm, and 3 μm (see, e.g., FIG. 5). The 12-μm micropillar array can be incorporated into the assay to trap potential large cell aggregates and cellular debris, whereas subsequent downstream arrays with progressively narrower openings between adjacent micropillars are configured to trap RBCs with increasing membrane stiffness. The microfluidic devices 208 and 210 can include one or more other sized micropillar arrays and/or other mechanisms configured to trap potential large cell aggregates and the like. The RBCs trapped in a given micropillar array or other trap mechanism would increase the magnitude of the electrical impedance measured between pairs of sensing electrodes (e.g., electrodes 122) located upstream and downstream of each micropillar array, enabling a rapid and fully electronic method for assessing RBC-mediated microvascular occlusion. To mimic arteriovenous anastomoses, passageways (e.g., approximately 40 μm-wide passageways) can be located on both sides of the micropillar arrays along the microchannel. These wider pathways can mitigate microchannel clogging by allowing the incoming cells to flow around already-occluded openings and interact with non-occluded openings downstream within the microchannel. The microfluidic devices 208 and 210 can have other numbers, sizes and arrangements of micropillar arrays in other examples.

The microfluidic devices 208 and 210 can be fabricated using lithography and/or other additive processing techniques, such as described in the above-incorporated PCT International Publication No. WO 2022/120139. The sensing electrodes can be substantially planar and formed of a conductive material (e.g., gold or another conductive material) on a surface of the microchannel spaced apart from each other in a direction of fluid flow on opposite sides of an occlusion region where each of the micropillar arrays resides (or will be formed) within the microchannel. The micropillar arrays can be arranged and configured in substantially parallel rows that extend perpendicular to the direction of fluid flow. Other arrangements and configurations of micropillars and sensing electrodes can be used in other examples.

In the example of FIG. 2, the analog and digital electronics 204 include a signal generator 212, an ADC 214, a digital I/O module 216, a processor 218, as well as other associated circuitry (e.g., memory, registers, digital-to-analog converters, interfaces, driver circuitry, etc.). In an example, the analog and digital electronics 204 can be implemented as an ADALM2000 software-defined instrument module (Analog Devices). As shown schematically in FIG. 2, the system 200 is based on an auto-balancing bridge topology, selected for its relative design simplicity and high accuracy over a wide range of impedances and measurement frequencies. For example, the signal generator 212 is configured to generate a 2-Vₚₚ, 10-kHz, sinusoidal excitation signal VIN, such as can be generated via direct digital synthesis by a 12b, 150-MSa/s digital-to-analog converter (DAC) in the mixed-signal front-end (M×FE) (AD9963; Analog Devices, Wilmington, MA) of the analog and digital electronics 204 (e.g., ADALM2000 software-defined instrument module). The 10-kHz operation frequency was selected as a tradeoff between the electrode polarization effects at lower frequencies and increased parasitic effects at higher frequencies. Other frequencies can be used in other examples.

The front-end interface circuitry 206 (also referred to as a front-end interface board) includes a demultiplexer 220, a multiplexer 222, and a transimpedance amplifier (TIA) 224. In an example, each of the demultiplexer 220 and the multiplexer 222 can be implemented by the CD74HCT4051 digitally controlled analog switch available from Texas Instruments, of Dallas, TX. The TIA 224 can be implemented by the AD8057 operational amplifier available from Analog Devices. A feedback impedance ZFB (e.g., a nominal resistance of approximately 100 k (2) can be coupled between the inverting input terminal and the output terminal of the TIA 224. Other circuitry can be used in other examples to implement the functions of the front-end interface circuitry 206. The analog and digital electronics 204 can include digital I/O terminals coupled to control inputs of the demultiplexer 220 and the multiplexer 222 to provide a number of digital control bits to control each of the demultiplexer and multiplexer (e.g., 3 bits to the multiplexer and 3 bits to the demultiplexer).

As an example, the excitation signal VIN can be routed by the demultiplexer 220 to one of the electrode contact pads on the one side (e.g., shown at the left side in FIG. 2) of each microfluidic device 208 and 210. Similarly, the analog multiplexer 222 has inputs coupled to the electrode contact pads on the other side (e.g., shown at the right side in FIG. 2) of each microfluidic device 208 and 210. The inputs of the multiplexer 222 selectively receive a corresponding response to the VIN, which the multiplexer can route to the input of the TIA 224. Using a total of six control bits, shown as bits D0 through D5, this example arrangement can permit the excitation signal to be successively routed through one selected electrode pair (and its associated micropillar array) at a time, thereby enabling multiplexed measurements across twelve micropillar arrays of two microfluidic devices 208 and 210. In the example of FIG. 2, the respective measurements are represented by impedances Z₁₁-Z₁₆ (for microfluidic device 208) and Z₂₁-Z₂₆ (for microfluidic device 210).

The TIA 224 can in turn provide the output signal VOUT to an input of the analog and digital electronics 204 in response to routing of the excitation signal VIN through a selected micropillar array. The output signal VOUT can be calculated according to the following equation:

$$VOUT = VIN \frac{{Z}_{F B}}{{Z}_{A}} \qquad ( 1 )$$

where Z_{A} is the impedance of the selected micropillar array (e.g., one of the impedances Z₁₁-Z₁₆ or Z₂₁-Z₂₆).

As an example, the signals VIN and VOUT can be sampled concurrently by two 12b, 100-MSa/s analog-to-digital converters (ADCs) in the M×FE. From Eq. 1, Z_{A} can be calculated as:

$${Z}_{A} = {Z}_{F B} \frac{VIN}{VOUT} \qquad ( 2 )$$

where VIN and VOUT are the ADC-sampled excitation and TIA output signals, respectively.

In addition to the front-end interface board 206 including the demultiplexing/multiplexing and amplification circuitry, it can also include a holder (e.g., a 3D-printed cradle) adapted to hold the microfluidic devices 208 and 210 (e.g., chips) during testing. Electrical connection between the contact pads of the chips and the demultiplexer/multiplexer lines can be made by a set of spring-loaded contact pins (e.g., available from Mill-Max, Oyster Bay, NY) mounted on three small boards. These boards connect to the front-end interface board 206 via fixed pin headers, aligning the contact pins with the contact pads on each microfluidic device 208 and 210 (e.g., MIRCA microfluidic chips) for a robust electrical and physical connection between the chips and the circuitry of the front-end interface board 206.

As a further example, the ADALM2000 software-defined instrument module, which is developed around a Zynq-7000 system-on-chip core (XC7Z010; Xilinx, San Jose, CA), can drive up to sixteen general-purpose input/output (GPIO) pins (e.g., via digital I/O block 216) for digital control, on-board signal conditioning, filtering, and temperature calibration, and enable the communication of data and programming signals with the computing apparatus 202.

The computing apparatus 202 can include a processor 230, memory 232, and a display 234. The display 234 can be implemented as an interactive touchscreen that provides an input device to a user interface 236 through which a user can enter commands and control operation of the system 200. The memory 232 can also include instructions 238 that, when executed by the processor 230, cause the processor to perform respective control logic, functions, and/or methods described herein. The memory can also store data, such as electrical measurement data, impedance data, and results of calculations performed by the processor.

In an example, the computing apparatus 202 can be implemented as a Raspberry Pi 4 computing module (e.g., available from Raspberry Pi Foundation, Cambridge, UK) with an attachable touchscreen display. For example, following the transfer of acquired signals by the analog and digital electronics 204 (e.g., implemented as the ADALM2000), impedance calculations for each micropillar array using Eq. 2 can be performed by Python-based software (e.g., the instructions 238) running on the Raspberry Pi (e.g., on processor 230 of computing apparatus 202). In some examples, each acquisition by the ADCs 214 can include a total of 216 samples, which can be trimmed to 50,000 samples by the Raspberry Pi for Fast Fourier Transform (FFT) calculations on the raw VIN and VOUT signals. This is intended to isolate the desired information at 10 kHz from broad-spectrum sources of noise. Moreover, a properly sized FFT calculation with an integer number of signal periods can ensure that the total energy around 10 kHz is contained within a single frequency bin. The computing apparatus 202 can be implemented by other computing architectures in other examples.

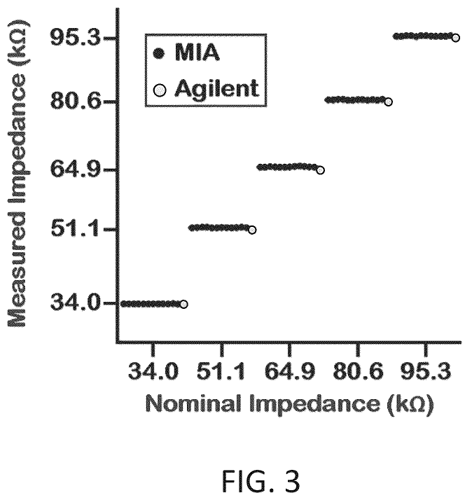

For the experimental results disclosed herein (see, e.g., FIGS. 3, 4, 6, and 8-13F), blood samples from healthy donors (HbAA) were obtained from the Hematopoietic Biorepository and Cellular Therapy Core at Case Western Reserve University under an institutional review board (IRB)—approved protocol. Samples from patients with homozygous (HbSS) SCD were obtained from the University Hospitals Cleveland Medical Center (UHCMC) under another IRB-approved protocol. Samples from treatment-naïve patients with HbSS SCD were obtained from the Joint Clinical Research Centre (JCRC) in Kampala, Uganda under an IRB-approved protocol at the JCRC. All samples were collected in ethyl-enediaminetetraacetic acid (EDTA)—containing vacutainers and stored at 4° C. until used. All experiments were performed within 48 h of sample collection. Furthermore, RBC hemoglobin profiles were obtained using the Bio-Rad Variant II Hemoglobin Testing System (Bio-Rad Laboratories, Hercules, CA) for samples obtained from UHCMC and with the Gazelle Hemoglobin Variant (Hemex Health, Portland, OR) for samples obtained from JCRC. Other clinical variables such as lactate dehydrogenase, absolute reticulocyte count, hematocrit, RBC count, total hemoglobin, and mean corpuscular volume were obtained from clinical records at the respective health facilities for use in the experiments.

As described herein, the fluid samples can include red blood cells. For example, to prepare each blood sample for testing, RBCs can be extracted from whole blood by centrifugation (e.g., at 500 g for 5 minutes), followed by removal of the plasma and the buffy coat. The RBC pellet can be washed with 1X phosphate-buffered saline (PBS) and centrifuged at 500 g for 5 minutes, with each step repeated twice. For experiments not involving glutaraldehyde, the RBCs can be re-suspended in 1×PBS at 20% hematocrit. The total sample preparation time was approximately 20 minutes. In other examples, the fluid sample can include a mixture of diseased (e.g., SCD) RBCs and healthy RBCs, whole blood, or other types of fluid samples. Also, or alternatively, the RBC or other fluid sample can include a therapeutic agent.

As a further example, for the duration of the RBC fluid sample and PBS perfusion steps (when PBS is utilized), impedance measurements on each micropillar array can be performed by the system 200 every 1.5 s and averaged to produce a single data point (e.g., impedance value) every 5 s. The impedance values for each array were written to a csv file on the Raspberry Pi's memory card (e.g., memory 232) and displayed to the user on display 234 in real-time via a running plot on the MIA system's graphical user interface 236.

The systems and methods described herein can be configured to implement a wash-based or wash-free assay. As an example, an occlusion index (OI) parameter can be defined for the MIRCA assay based on electrical impedance measurements performed by the MIA (e.g., the system 100, 200). For the example of a wash-based assay operation (e.g., having micropillar openings of 12 μm, 10 μm, 8 μm, 6 μm, 4 μm, and 3 μm), the OI parameter can be calculated according to the following equations:

$${OI}_{w a s h} = \frac{{Z}_{3} \cdot {N}_{3} + … + {Z}_{10} {N}_{10}}{{N}_{3} + … + {N}_{10}} \qquad ( 3 )$$

$$where :$$

$${Z}_{n} = \frac{{Z}_{n , RBC}}{{Z}_{n , baseline}} \qquad ( 4 )$$

Zₙ represents the normalized impedance change determined for the n-μm micropillar array,
Nₙ represents a total number of openings in the n-μm micropillar array,
Zₙ, baseline represents a baseline impedance of the n-μm micropillar array measured before the fluid sample enters the microchannel, and
Zₙ, RBC represents the impedance of the n-μm micropillar array measured after the fluid sample is flowing through each of the plurality of micropillar arrays of the microchannel.

As a further example, there can be approximately 9945, 11067, 12444, 13260, 14229, and 15300 openings in the 12 μm, 10 μm, 8 μm, 6 μm, 4 μm, and 3 μm arrays, respectively. Other numbers of openings can be used in the 12 μm, 10 μm, 8 μm, 6 μm, 4 μm, and 3 μm arrays in other examples. For the OI calculations, only the 3, 4, 6, 8, and 10-μm micropillar arrays were considered, since impedance variations in the 12-μm micropillar array were largely attributed to cellular debris and large cell aggregates as described herein.

For wash-free assay operation for microfluidic devices having 12 μm, 10 μm, 8 μm, 6 μm, 4 μm, and 3 μm micropillar arrays, an alternative OI parameter OI_{wash-free} can be defined and calculated as follows:

$${OI}_{w ash - free} = \frac{( {Z}_{3} - {Z}_{10} ) \cdot {N}_{3} + … + ( {Z}_{10} - {Z}_{10} ) \cdot {N}_{10}}{{N}_{3} + … + {N}_{10}} \qquad ( 5 )$$

where:
Zₙ represents the normalized impedance change of the n-μm micropillar array as defined in Eq. 4, but with Zₙ. RBC being measured after only about 10 minutes of RBC sample perfusion and with no PBS wash step for the microchannel where measurements are being performed.
   In other wash-free examples, Zₙ, RBC can be measured after waiting for sample perfusion to occur for a different amount of time.

The wash-free OI for the RBC sample in Eq. 5 further can be generalized for micropillar arrays having other sizes and numbers and configurations of micropillar arrays, in which the OI_{wash-free} can be calculated according to the following equation:

$${OI}_{w ash - free} = \frac{( {Z}_{1} - {Z}_{j} ) \cdot {N}_{1} + ( {Z}_{2} - {Z}_{j} ) \cdot {N}_{2} + … + ( {Z}_{m} - {Z}_{j} ) \cdot {N}_{m}}{{N}_{1} + {N}_{2} … + {N}_{m}} \qquad ( 6 )$$

$$where :$$

$${Z}_{n} = \frac{{Z}_{n , RBC}}{{Z}_{n , baseline}}$$

Zₙ represents the normalized impedance change determined for an n^{th} micropillar array, where n and j are positive integers from 1 to m, and m+1 represents a total number of the micropillar arrays in the microchannel,
Nₙ represents a total number of openings in the n^{th} micropillar array,
Z_{n,baseline} represents a baseline impedance of the n^{th} micropillar array measured before the fluid sample enters the microchannel, and
Z_{n,RBC} represents the impedance of the n^{th} micropillar array measured after waiting for the fluid sample to flow through each of the plurality of micropillar arrays of the microchannel for a period of time (e.g., 2 minutes through 15 minutes, such as 10 minutes or less).

FIG. 3 is a graph depicting a comparison of impedances measured by an example MIA (e.g., system 100, 200) and a benchtop impedance analyzer. For example, FIG. 3 shows measured impedance values versus those measured by a benchtop Keysight Technologies 4294A impedance analyzer for five nominal, fixed, test impedances in the range of approximately 34-95 kΩ. Root mean square (RMS) deviation for the MIA system and Keysight measurements over this range was ≤0.6% for all twelve measurement channels. FIG. 3 thus shows excellent agreement between the two systems, demonstrating a high level of accuracy for the MIA (e.g., system 100, 200).

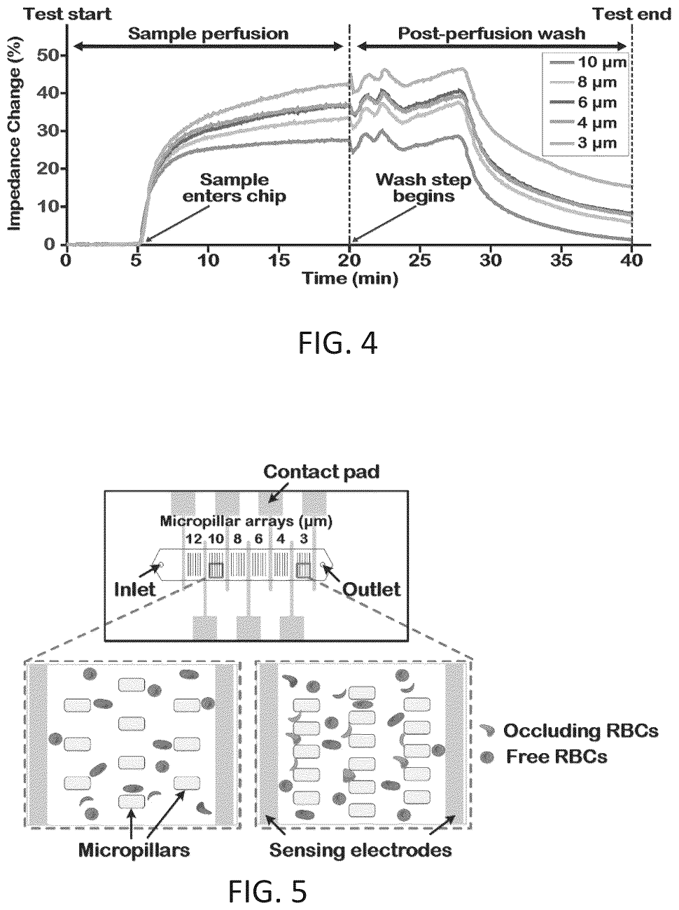

FIG. 4 is a graph of measured impedance curves representing impedance values for a number of different micropillar arrays normalized to baseline values (e.g., according to Eq. 4), shown as percentage variations, depicting impedance changes over time. The impedance measurements were performed over time following an initial perfusion step with PBS for fluid samples containing RBCs reconstituted in 1X PBS at 20% hematocrit that were introduced into the microfluidic device (e.g., device 108, 208, 210). The 12-μm micropillar array was not included in data analysis and was omitted from the graph of FIG. 4. This is because the 12-μm micropillar array is incorporated into the assay to trap potential large cell aggregates that may otherwise cause microchannel clogging.

FIG. 4 shows a sharp increase in the impedance measured across each micropillar array at the time point when the sample entered the microfluidic chip. During the first few minutes of sample perfusion, impedance changes were primarily the result of free, non-occluding RBCs flowing through each array and displacing an equivalent volume of PBS. As sample perfusion continued, a greater number of RBCs became trapped by the micropillar arrays, increasing the concentration of RBCs localized to each array and thus increasing the respective impedance levels. The relative contribution of occluding and non-occluding RBCs to the total impedance is determined by the opening size of each micropillar array, with arrays with larger openings having fewer RBC-mediated occlusions. For example, the impedance change across the 10-μm micropillar array can be almost entirely attributed to free, non-occluding RBCs. As such, the impedance change across this array leveled off at a faster rate than that of the arrays with smaller openings. Additionally, the post-perfusion PBS wash step resulted in a sharp decrease in the impedance measured across each micropillar array as free, non-occluding RBCs were washed out, leaving mostly (or only) occluding RBCs within the microchannel.

As described herein, the systems and methods described herein can compensate for the impedance contribution resulting from free non-occluding red blood cells and/or other objects present in the microchannel. For example, FIG. 5 is a schematic diagram showing RBCs flowing through micropillar arrays of a microchannel. As described herein, the raw impedance change at each micropillar array represents the combined contributions of free and occluding RBCs. For example, as seen in Eqs. 5 and 6 and illustrated in FIG. 5, to measure the effect of occluding RBCs only in a wash-free assay, the normalized impedance change of the 10-μm micropillar array (or other array where minimal occlusion is expected to occur) is subtracted from that of the other arrays, effectively subtracting the contribution of free, non-occluding RBCs present in the microchannel during the sample perfusion period. The weighted summation of these changes over each array thus represents the fractional occlusion of the whole MIRCA microfluidic device. This obviates the need for a PBS wash step to physically remove the free, non-occluding RBCs, significantly reducing the assay run time (e.g., by about 62.5%, or from about 40 minutes to about 15 minutes) and the complexity of the testing protocol for the user. Advantageously, the systems and methods herein can be implemented in a wash-free assay.

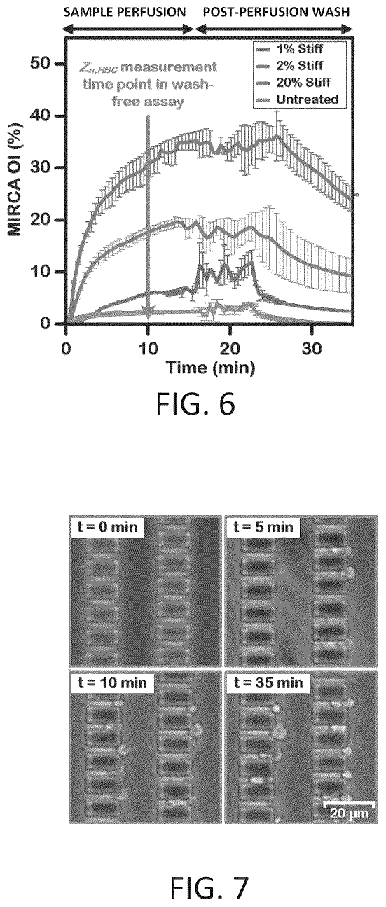

To validate the functionality of the MIRCA wash-free assay, samples containing 0% (i.e., untreated), 1%, 2%, and 20% glutaraldehyde-treated membrane-stiffened RBCs were initially tested under standard wash-based assay conditions. FIG. 6 is a graph depicting examples of occlusion indexes (shown as MIRCA OIs) for different RBC samples observed before and after a wash step. In this example, an impedance-measurement time point of 10 minutes is chosen to calculate the wash-free assay OI based on tradeoffs among run time, sample discrimination, and correlation to the optically measured OI. The time t=0 minutes corresponds to when the samples enter the microfluidic chip. As seen in FIG. 6, samples with higher fractions of membrane-stiffened RBCs yielded a sharper rise in the MIRCA OI parameter tracked throughout the duration of the experiment, with a clear separation among the various treatment groups visible throughout the perfusion period. From this data, the MIRCA post-wash OI (based on Eq. 3 with the impedance of each micropillar array measured at the conclusion of the 40-minute run time of the assay) and the MIRCA wash-free OI (based on Eq. 5 with the impedance of each micropillar array measured after only 10 minutes of RBC sample perfusion) were calculated. An optical OI, representing the overall percentage of a microcapillary network occluded by the RBCs, was also obtained in each experiment.

FIG. 7 depicts microscopic images of micropillar arrays in a microchannel at different times during sample perfusion through the microchannel. Specifically, as seen in FIG. 7, microscopic images of the microchannel were taken at three different time points (t=0, 5, 10 minutes) during sample perfusion to visually observe the gradual occlusion of micropillar openings by membrane-stiffened RBCs, as well as at a final time point of t=35 minutes to see the occluding RBCs that remained after the post-perfusion wash step. These time points are referenced to when the sample first entered the microfluidic chip after approximately 5 minutes of travel time from the sample reservoir to the microchannel. Moreover, the exposure time on the microscope was increased to 300 ms to blur the free, non-occluding RBCs moving through the microchannel, leaving only stationary, occluding RBCs visible. Optical Ols were calculated by manually counting the number of occluded micropillar openings in the images taken at t=35 minutes.

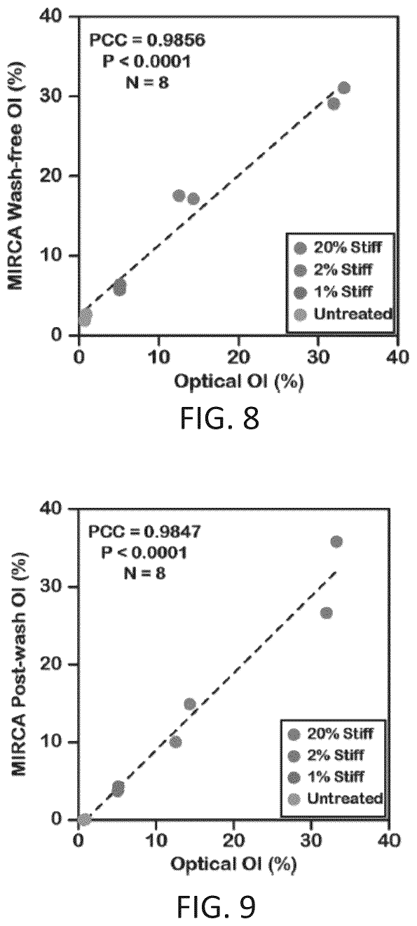

FIGS. 8 and 9 are graphs plotting correlation between OIs determined for wash-free and post-wash MIRCA assays, respectively, relative to optical OIs determined for a range of fluid samples. The occlusion indexes of the wash-free (FIG. 8) and post-wash (FIG. 9) MIRCA assays exhibit very strong positive correlations to the optically obtained values. Specifically, FIG. 8 shows a very strong, positive, linear correlation (PCC=0.9856, P<0.0001, N=8) between the MIRCA wash-free and optical OI parameters, whereas FIG. 9 shows the same level of linear correlation (PCC=0.9847, P<0.0001, N=8) between the MIRCA post-wash and optical OI parameters. Collectively, these data validate the functionality of the MIRCA wash-free assay that in turn significantly reduces the runtime of the assay and complexity of the protocol.

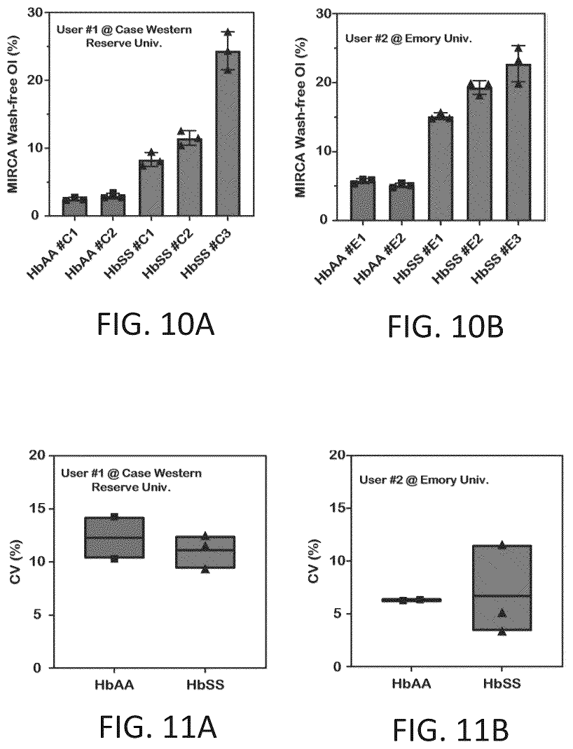

FIGS. 10A and 10B are bar graphs depicting examples of impedance-based occlusion indexes determined for a wash-free assay for different blood cell samples assessed by different users at different locations. FIGS. 11A and 11B are graphs depicting floating bars showing variations among the users at each location for the same wash-free MIRCA assay. In these examples, the MIRCA wash-free assay was assessed by two different trained users at two separate locations, namely, a research laboratory at Case Western Reserve University and a research laboratory at Emory University School of Medicine. Specifically, at each location, two healthy (HbAA) and three SCD (HbSS) samples were tested, each on three different MIRCA microfluidic chips. Means and standard deviations (SD) of the OI parameter (in %) are shown for each sample in FIGS. 10A and 10B. As expected, OI values obtained in each testing location were higher for HbSS as compared to HbAA samples. FIGS. 10-11 thus demonstrate the reproducibility of the MIRCA wash-free assay.

The range of coefficients of variation (CVs) for each participant group at each location is shown in FIGS. 11A and 11B. The midline represents the mean CV and the individual data points indicate the CV for each sample. The mean CV at Case Western Reserve University was about 12.3% for HbAA and about 11.1% for HbSS samples. At Emory University, the mean CV was about 6.3% for HbAA and about 6.7% for HbSS samples. Importantly, all CV values across the two users and testing locations were within the precision criterion of 20% established in Bioanalytical Method Validation guidelines of the FDA.

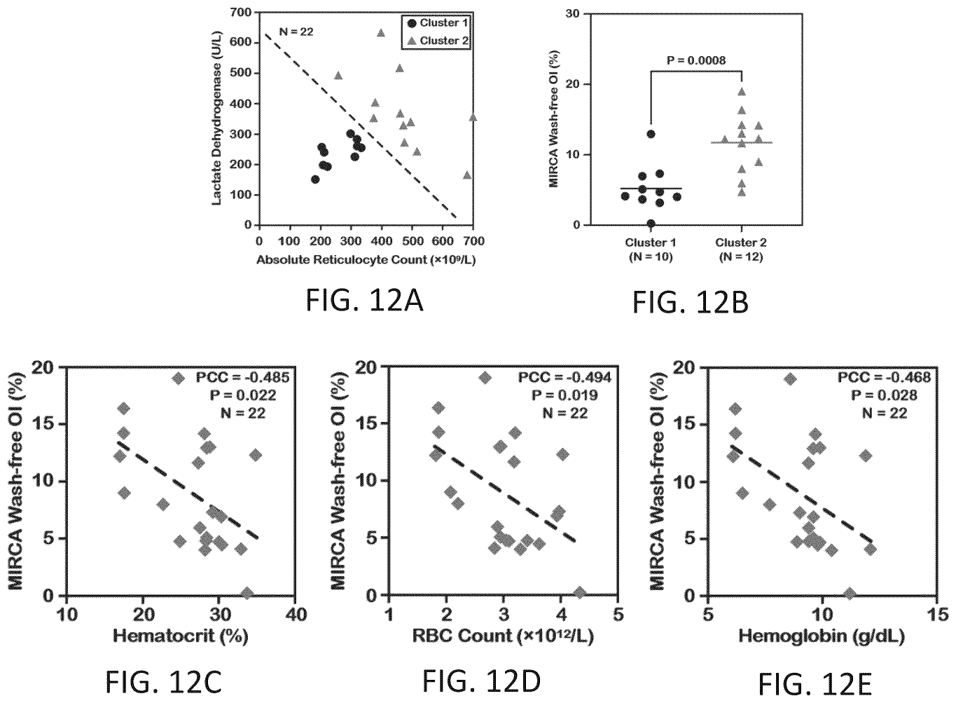

FIGS. 12A-12E are graphs depicting a correlation between the wash-free occlusion index and biomarkers of RBC hemolysis, further demonstrating the clinical utility of the systems and methods described herein. The data provided in FIGS. 12A-12E evaluate the relationship between the OI parameter and conventional laboratory measurements of RBC hemolysis (e.g., lactate dehydrogenase (LDH) and absolute reticulocyte count (ARC)) in 22 patients with homozygous (HbSS) SCD.

For the graph of FIG. 12A, samples from 22 patients with homozygous (HbSS) SCD were classified into two clusters using K-means clustering based on their lactate dehydrogenase (LDH) level and absolute reticulocyte count (ARC). As seen in FIG. 12A, the first cluster (N=10) had, in general, lower LDH and ARC, whereas the second cluster (N=12) generally had higher LDH and ARC. LDH and ARC are common biomarkers of elevated RBC hemolysis, given that LDH is released by RBCs upon rupture and that increased RBC rupture leads to increased production of reticulocytes by hematopoietic cells. Since high levels of hemolysis are associated with a more severe clinical course, these results confirmed expectations that samples with higher levels of these two biomarkers would yield higher levels of the OI parameter when measured with the MIRCA assay (e.g., implemented by systems 100, 200, or by method 300).

As seen in the graph of FIG. 12B, patients in the second cluster (N=12) had significantly higher OIs, with a mean±SD of 11.73%±4.18% compared to 5.21%±3.35% in the first cluster (N=10). Furthermore, a significant inverse relationship was observed between the OI and hematocrit (shown in FIG. 12C), RBC count (shown in FIG. 12D), and hemoglobin level (shown in FIG. 12E). Decreased levels of these biomarkers are also linked to RBC hemolysis and indicate a more severe case of sickle cell anemia. The P value in FIG. 12B was obtained using the Mann-Whitney nonparametric U test. Data in FIGS. 12C-12E were analyzed using the two-tailed t-test. Overall, these results are consistent with those in previous studies and support the clinical utility of the MIRCA wash-free OI as an indicator of RBC health and function.

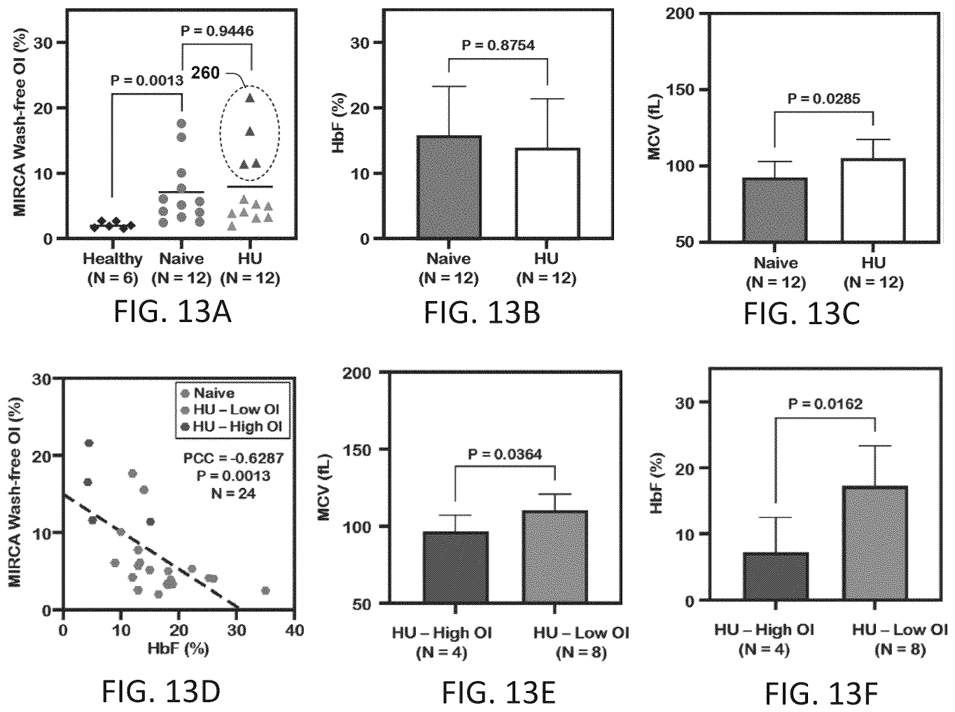

As a further example, FIGS. 13A-13F include graphs demonstrating experimental results and correlations based on measurements made using a prototype MIRCA assay. For example, a prototype MIRCA assay was deployed at the JCRC in Kampala, Uganda and utilized to measure RBC samples from Ugandan patients with homozygous (HbSS) SCD who had never received any SCD-specific therapeutic interventions (i.e., treatment-naïve). As seen in FIG. 13A, the OI parameter was significantly higher for these samples (N=12) compared to healthy (HbAA) samples (N=6) from the U.S. population (P=0.0013), with values (mean±SD) of about 7.03%±4.98% versus about 2.10%±0.50%, respectively. High heterogeneity was observed in the treatment-naïve group, with the OI parameter ranging from about 2.45% to about 17.63%. Interestingly, there was no statistically significant difference in the OI parameter between treatment-naïve samples from the Ugandan population and samples (N=12) from SCD patients on hydroxyurea (HU) treatment obtained in the U.S. (P=0.9446). This similarity in OI may be due to the similarity in the percent fetal hemoglobin (% HbF), as there was no statistically significant difference in % HbF either between the treatment-naïve (15.8%+7.47%) and HU-treated (13.9%±7.43%) groups (P=0.8754; see FIG. 13B). The similarity in OI may also represent a survival bias, with milder phenotypes/higher endogenous % HbF selected for in Uganda given the high early childhood mortality rates compared to those in the U.S. It was previously shown that the OI parameter is lower in SCD patients who have high HbF levels. However, the patients in these previous studies were all on HU, which is known to have other effects on RBCs that may impact their deformability such as an increase in the mean corpuscular volume (MCV). It has been determined that the treatment-naïve patients had a significantly lower MCV than those on HU (P=0.0285; FIG. 13C) and that the OI parameter had a significant inverse relationship with % HbF (PCC=−0.6287, P=0.0013, N=24) across all treatment-naïve and HU-treated patients, as seen in FIG. 13D. The fact that OI is modulated by % HbF even in treatment-naïve patients shows that elevated % HbF can be sufficiently protective against microcapillary occlusion for both treatment-naïve and HU-treated SCD patients. This also highlights the relevance of % HbF specifically as no difference in the cohorts was noted despite the significantly higher MCV in the HU-treated patients. HbF prevents the extension of HbS polymer, allowing time for the RBCs to escape from the microcirculation back to the lungs to re-oxygenate rather than sickling and becoming trapped. Several long-term longitudinal studies have shown that SCD patients with elevated HbF levels experience a reduction in clinical complications and a lower frequency of vaso-occlusive crises (VOCs). The systems (e.g., system 100, 200) and methods (e.g., method 300) described herein thus can provide a useful assay in evaluating the therapeutic effect of HbF-inducing agents such as HU on RBC-mediated microvascular occlusion.

As a further example, the systems and methods described herein (e.g., the MIRCA wash-free assay) can be used to identify and evaluate differences in treatment outcomes (e.g., HU treatment outcomes). RBC samples from twelve SCD patients on HU were assessed with the MIRCA wash-free assay to determine if the OI parameter could be used to identify patients for whom HU treatment did not sufficiently mitigate their risk of microvascular occlusion. HU has been widely shown to improve RBC health in SCD by increasing MCV and HbF levels, making these biomarkers ideal candidates for evaluating HU treatment response. Notably, differences in HU treatment outcomes may result from resistance to HU, a lack of medication adherence, or suboptimal dosages below the patient's maximum tolerable dose.

Using the median absolute deviation method, four patients (circled at 260 in FIG. 13A) were identified as outliers with very high OI levels, permitting the identification of two patient subgroups, namely, HU-Low OI (N=8) and HU-High OI (N=4). We found that patients in the HU-High OI subgroup had significantly lower MCV (P=0.0364; FIG. 13E) and % HbF (P=0.0162; FIG. 13F) than those in the HU-Low OI subgroup, suggesting that classification based on the MIRCA OI parameter can identify patients who may need treatment adjustments to improve their outcomes.

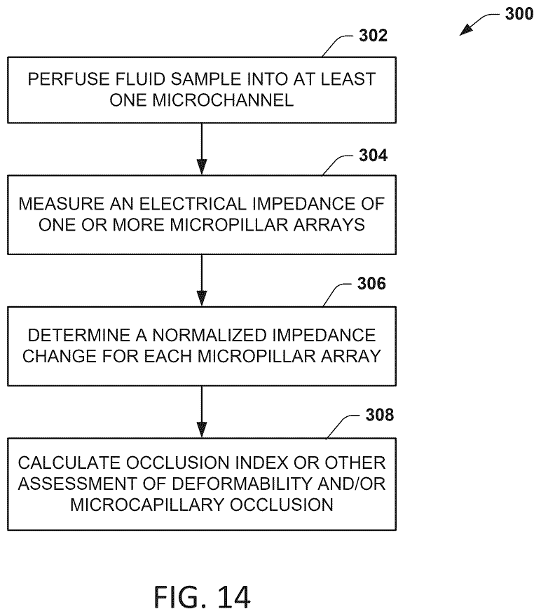

In view of the foregoing structural and functional features described above, example methods that can be implemented will be better appreciated with reference to the flow diagram of FIG. 14. While, for purposes of simplicity of explanation, the method 300 of FIG. 14 is shown and described as executing serially, it is to be understood and appreciated that the method is not limited by the illustrated order, as some aspects could, in other examples, occur in different orders and/or concurrently. Moreover, not all illustrated features may be required to implement a method. The method 300 or portions thereof can be implemented as instructions stored in one or more non-transitory machine readable media (e.g., memory 144, 232) and be executed by a processor (e.g., processor 142, 230, 218) of one or more computing apparatuses, for example. The method 300 can be implemented by the systems described herein, including FIGS. 1-2. Accordingly, the method 300 may refer to certain aspects of FIGS. 1-7.

At 302, the method includes perfusing a fluid sample including red blood cells into at least one microchannel of a microfluidic device (e.g., microfluidic device 112, 208, 210 of FIGS. 1 and 2). The perfusion of the fluid sample can be controlled by a processor or microcontroller (e.g., processor 142, 218, 230), such as to achieve a desired flow rate and/or pressure. As described herein, the microchannel includes a plurality of micropillar arrays (e.g., arrays 120 of FIG. 1, see also FIGS. 5 and 7) arranged in a direction of fluid flow through the microchannel, and each of the plurality of micropillar arrays is located between a respective electrode pair (e.g., electrodes 122, see also FIG. 5). Each of the micropillar arrays can have progressively narrower micropillar openings in the direction of fluid flow.

At 304, the method includes measuring (e.g., by impedance analyzer 104-106, 202-206) an electrical impedance of one or more micropillar arrays, each of which is located between a respective electrode pair. The impedance measurements at 304 can include one or more measurements before the fluid sample has entered the microchannel (before perfusion of the fluid sample at 302) and one or more measurements after the fluid sample has entered the microchannel (e.g., responsive to the perfusion of the fluid sample at 302). For example, electrical impedance can be measured by providing an excitation signal (e.g., from signal generator 132, 212) to a first electrode of the respective electrode pair for a given one of the micropillar arrays during each measurement interval. An output signal can be measured at a second electrode of the respective electrode pair for the given one of the micropillar arrays responsive to the excitation signal during the measurement interval. The routing of the excitation signal to each pair of electrodes can be through a switch network (e.g., switch network 130, 220, 222) to successively route the excitation signal through each electrode pair during pre- and intra-perfusion measurement intervals, such as described herein. The impedance measurement can be determined (e.g., by processor 142, 230) for the micropillar array based on the excitation signal and the output signal (e.g., according to Eq. 2).

At 306, the method includes determining (e.g., by processor 142, 230) an impedance change (e.g., a normalized impedance change, such as in Eq. 4) for each of the micropillar arrays based on recorded measurements of electrical impedance (at 304). As described herein, the normalized impedance change can be determined based on a baseline impedance measurement performed before perfusion of the fluid sample through the microchannel and an impedance measurement performed during perfusion of the fluid sample.

At 308, the method includes determining (e.g., by processor 142, 230) an assessment of deformability and/or microcapillary occlusion of the red blood cells in the fluid sample based on the impedance change determined (at 306) for the micropillar arrays. For example, the assessment can be calculated as an occlusion index parameter (see, e.g., Eqs. 3, 5, and 6). Also, in some examples, the assessment of deformability and/or microcapillary occlusion can be calculated at 308 to compensate for a contribution of free non-occluding red blood cells and/or other objects present in the microchannel. For example, the method 300 can include estimating an impedance contribution caused by free non-occluding red blood cells present in the microchannel and subtracting the estimated impedance contribution from the impedance change determined (at 306) for the micropillar arrays (see, e.g., Eqs. 5 and 6).

Also, or alternatively, the perfusion of the fluid sample (at 302) and the measuring of electrical impedance (at 304) can be performed in the absence of washing of the microchannel (e.g., in a wash-free assay). In other examples, the method 300 can be performed in a wash-based assay.

Also, in some examples, the method 300 can be performed to measure the efficacy of a therapeutic agent in modulating blood cell adhesion and/or deformability. The therapeutic agent can be added to the fluid sample prior to perfusion through the microchannel. Also, or alternatively, a therapeutic agent can be administered to a subject (as a treatment or therapy) to modulate blood cell adhesion and/or deformability of RBCs in vivo, and the fluid sample being perfused (at 302) can include RBCs from the subject's blood that has been modulated by the therapeutic agent. The measure of efficacy of the therapeutic agent can be determined based on comparing the impedance change (normalized impedance change) for one or more micropillar arrays to a control. Also, or alternatively, the measure of efficacy for the therapeutic agent further can be determined based on comparing an OI determined for a number of micropillar arrays to a control. The control can be an impedance change or an OI that was determined (by the method 300) for a fluid sample without the therapeutic agent or a fluid sample that includes a different amount of the agent. For example, a decrease in the measured impedance change and/or OI compared to a control (e.g., an impedance measurement and/or OI for a sample without the agent or a different amount of the agent) is indicative of the therapeutic agent having an increased efficacy in decreasing blood cell adhesion and/or increasing blood cell deformability.

Currently, several novel gene therapies are being developed to increase HbF levels as a potential one-time cure for SCD. Assessment of the effectiveness of these new therapies is conventionally done by tracking clinical indicators such as the frequency of VOCs, which requires lengthy, longitudinal monitoring of the patients. The MIRCA assay (e.g., implemented by systems and methods herein), with its rapid electrical impedance-based readout scheme, offers a promising solution for accelerated evaluation of RBC health, function, and therapeutic effect in an ex vivo model of the microcapillary networks. Such an alternative solution for effectiveness assessment would greatly benefit the ongoing efforts focused on developing curative therapies for SCD.

Additionally, as shown herein, an association exists between OI and known biomarkers of disease severity such as LDH, ARC, and % HbF. It has also been shown that OI changes with known disease-modifying therapies such as HU. It is further expected that OI correlates with rates of clinical complications, and therapy-related reduction in OI is associated with clinical improvement.

The systems and methods described herein thus provide for accelerated evaluation of RBC health, function, and therapeutic effect in an ex vivo model of the microcapillary networks. For example, the occlusion of micropillar structures with progressively narrower openings along the direction of fluid flow by RBCs of variable stiffness can be measured in real-time by an MIA (e.g., system 100, 200) as the electronic readout system of the assay. The MIRCA assay can further employ a wash-free operation to enable a short run time of 15 minutes or less for sample perfusion with reduced complexity of the testing protocol for the user. The OI parameter of HbSS SCD patients further shows a significant positive relationship with several indicators of disease severity such as lowered hematocrit, hemoglobin level, RBC count, and % HbF. Patients with high levels of the hemolytic biomarkers LDH and ARC were also found to have significantly elevated OIs. Moreover, the OI parameter can be used to identify heterogeneity in HU treatment outcomes not detectable by conventional laboratory testing.

Also, or alternatively, the microfluidic devices, systems, and methods described herein can be used to assess small changes in RBC deformability, under both normoxic (ambient air) and hypoxic conditions, to assess pathologically impaired RBCs in blood. The assessment of pathologically impaired RBCs can be used to assess microvascular health and function of a subject and determine the subject's increased risk of vaso-occlusive crises (VOCs) in a range of microcirculatory diseases.

What have been described above are examples. It is, of course, not possible to describe every conceivable combination of components or methods, but one of ordinary skill in the art will recognize that many further combinations and permutations are possible. Accordingly, the invention is intended to embrace all such alterations, modifications, and variations that fall within the scope of this application, including the appended claims. Where the disclosure or claims recite “a,” “an,” “a first,” or “another” element, or the equivalent thereof, it should be interpreted to include one or more than one such element, neither requiring nor excluding two or more such elements.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device or system.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media (e.g., memory 144, 232, etc.) may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a processor).

Instructions may be executed by one or more processors, such as one or more digital signal processors (DSPs), general purpose microprocessors, application specific integrated circuits (ASICs), field-programmable gate arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

Furthermore, a circuit or device that is said to include certain components may instead be configured to couple to those components to form the described circuitry, device, or system. For example, a structure described as including one or more elements A, B and C may instead include only the A elements within a single physical device and may be configured to couple to at least some of the elements B and/or C to form the described circuitry, device, or system, either at a time of manufacture or after a time of manufacture, for example, by an end-user and/or a third-party.

As used herein, the term “includes” means includes but not limited to, the term “including” means including but not limited to. The term “based on” means based at least in part on. Also, in this description, numerical designations “first”, “second”, etc. are not necessarily consistent with same designations in the claims herein and these numerical designations are used to simply distinguish one element from another.

Additionally, the term “couple” or variants thereof may cover connections, communications, or signal paths that enable a functional relationship consistent with this description. For example, if device A generates a signal to control device B to perform an action, then: (a) in a first example, device A is directly coupled to device B; or (b) in a second example, device A is indirectly coupled to device B through intervening component C if intervening component C does not alter the functional relationship between device A and device B, so device B is controlled by device A via the control signal generated by device A. In this description, the term “based on” means based at least in part on.

Also, in this description, a device that is “configured to” perform a task or function may be configured (e.g., programmed and/or hardwired) at a time of manufacturing by a manufacturer to perform the function and/or may be configurable (or reconfigurable) by a user after manufacturing to perform the function and/or other additional or alternative functions. The configuring may be through firmware and/or software programming of the device, through a construction and/or layout of hardware components and interconnections of the device, or a combination thereof.

Also, the claims may be drafted to exclude any optional element. As such, this statement is intended to serve as antecedent basis for use of such exclusive terminology as “solely”, “only” and the like in connection with the recitation of claim elements, or the use of a “negative” limitation.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

We claim:

1. A system for wash-free electrical impedance-based assessment of blood cell properties comprising:
a microfluidic device that includes a microchannel extending through a portion of a housing, the microchannel being configured to receive a fluid sample including blood cells that flows along in a direction of fluid flow through the microchannel, the microchannel including a plurality of micropillar arrays along the direction of fluid flow, and each of the plurality of micropillar arrays being located between a respective pair of electrodes in the microchannel; and
an impedance analyzer coupled to each of the electrodes and configured to measure electrical impedance of at least some of the micropillar arrays at multiple times, in a wash-free assay, including at least one impedance measurement before the fluid sample is flowing through the microchannel and at least one impedance measurement after the fluid sample is flowing through the microchannel.

2. The system of claim 1, wherein the impedance analyzer comprises:
a signal generator configured to generate an excitation signal;
a switch network configured to route the excitation signal to a selected electrode of a given pair of the electrodes to pass the excitation signal through a respective one of the micropillar arrays to another electrode of the given pair of electrodes and provide a corresponding signal to an output of the switch network; and
an amplifier configured to provide an output signal responsive to the corresponding signal, wherein the impedance analyzer is configured to determine the measure of electrical impedance based on the excitation signal and the output signal.

3. The system of claim 2, wherein the switch network comprises:
a demultiplexer having an input and a plurality of outputs, wherein the input of the demultiplexer is coupled to an output of the signal generator to receive the excitation signal, the plurality of outputs is coupled to an input electrode of each respective pair of the electrodes, and the demultiplexer is configured to route the excitation signal to the input electrode of each respective pair of the electrodes responsive to a first selection signal; and
a multiplexer having a plurality of inputs and an output, wherein each of the plurality of inputs of the multiplexer is coupled to an output electrode of each respective pair of the electrodes, the output of the multiplexer is coupled to an input of the amplifier, and the multiplexer is configured to route the corresponding signal from a selected one of the plurality of inputs to the output of the multiplexer responsive to a second selection signal.

4. The system of claim 3, wherein the impedance analyzer comprises:
a processor configured to provide the first selection signal and the second selection signal to coordinate operation of the demultiplexer and the multiplexer in a time-multiplexed sequence such that the excitation signal is permitted to flow through a single pair of the electrodes and one of the micropillar arrays at a time.

5. The system of claim 2, wherein the amplifier comprises a transimpedance amplifier.

6. The system of claim 1, wherein the fluid sample includes red blood cells, and the impedance analyzer further comprises:
a processor configured to determine a normalized impedance change for the at least some of the micropillar arrays based on the impedance measurements for each of the at least some of the micropillar arrays before and after the fluid sample is flowing through the microchannel.

7. The system of claim 6, wherein the processor is further configured to:
estimate a normalized impedance contribution caused by free non-occluding red blood cells present in the microchannel; and
subtract the normalized impedance contribution from the normalized impedance change determined for each of the at least some of the micropillar arrays to provide a corresponding compensated normalized impedance change for each of the at least some of the micropillar arrays.

8. The system of claim 7, wherein the processor is further configured to determine an assessment of deformability and/or microcapillary occlusion of the red blood cells in the fluid sample based on the compensated normalized impedance change for the at least some of the micropillar arrays.

9. The system of claim 8, wherein the processor is configured to calculate an index indicative of the assessment of deformability and/or microcapillary occlusion of the red blood cells in the fluid sample based on the normalized impedance change determined for the at least some of the micropillar arrays.

10. The system of claim 9, wherein the index defines an occlusion index (OI) for the red blood cells in the fluid sample, and the processor is configured to calculate the OI according to the following equation:
$$OI = \frac{( {Z}_{1} - {Z}_{j} ) \cdot {N}_{1} + ( {Z}_{2} - {Z}_{j} ) \cdot {N}_{2} + … + ( {Z}_{m} - {Z}_{j} ) \cdot {N}_{m}}{{N}_{1} + {N}_{2} … + {N}_{m}}$$
$$where :$$
$${Z}_{n} = \frac{{Z}_{n , RBC}}{{Z}_{n , baseline}}$$
Zₙ represents the normalized impedance change determined for an n^{th} micropillar array, where n and j are positive integers from 1 to m, and m+1 represents a total number of the micropillar arrays in the microchannel,
Nₙ represents a total number of openings in the n^{th} micropillar array,
Z_{n,baseline} represents a baseline impedance of the n^{th} micropillar array measured before the fluid sample enters the microchannel, and
Z_{n,RBC} represents the impedance of the n^{th} micropillar array measured after the fluid sample is flowing through each of the plurality of micropillar arrays of the microchannel.

11. The system of claim 1, wherein the fluid sample includes a therapeutic agent to modulate blood cell adhesion and/or deformability.

12. A method comprising:
perfusing a fluid sample including red blood cells into at least one microchannel of a microfluidic device, wherein the at least one microchannel includes a plurality of micropillar arrays arranged in a direction of fluid flow through the at least one microchannel, and each of the plurality of micropillar arrays is located between a respective electrode pair;
measuring an electrical impedance of at least one micropillar array between a respective electrode pair including at least one measurement before the fluid sample has entered the microchannel and at least one measurement after the fluid sample has entered the microchannel, wherein the perfusion of the fluid sample and the measuring of electrical impedance are performed in the absence of washing of the microchannel;
determining a normalized impedance change for the at least one micropillar array based on the measurements of the electrical impedance; and
determining an assessment of deformability and/or microcapillary occlusion of the red blood cells in the fluid sample based on the normalized impedance change determined for the at least one micropillar array.

13. The method of claim 12, wherein the plurality of micropillar arrays are arranged in a direction of fluid flow through the microchannel, each of the plurality of micropillar arrays having progressively narrower micropillar openings in the direction of fluid flow,
wherein the electrical impedance is measured for at least some of the micropillar arrays at times that include before and after the fluid sample has entered the microchannel,
wherein normalized impedance changes are determined for the at least some of the micropillar arrays based on the electrical impedance measured between the respective electrode pairs, and
wherein the assessment of deformability and/or microcapillary occlusion is determined based on the normalized impedance changes determined for the at least some of the micropillar arrays.

14. The method of claim 12, wherein measuring the electrical impedance of the at least one micropillar array comprises:
measuring a first impedance of the at least one micropillar array before the fluid sample has entered the at least one micropillar array, wherein the first impedance defines a baseline impedance measurement; and
measuring a second impedance of the at least one micropillar array after the fluid sample has entered the at least one micropillar array for at least a predetermined period of time, wherein the normalized impedance change is determined for the at least one micropillar array based on the first impedance and the second impedance.

15. The method of claim 12, wherein measuring the electrical impedance comprises:
applying an excitation signal to a first electrode of the respective electrode pair for a given one of the micropillar arrays during a measurement interval;
receiving an output signal at a second electrode of the respective electrode pair for the given one of the micropillar arrays responsive to the excitation signal during the measurement interval; and
determining the measurements of the electrical impedance for the given one of the micropillar arrays based on the excitation signal and the output signal.

16. The method of claim 12, wherein the plurality of micropillar arrays comprises a first micropillar array and a second micropillar array within the microchannel located adjacent to each other along a direction of fluid flow through the microchannel, the first micropillar array is located between a first electrode and a second electrode, defining a first electrode pair, and the second micropillar array is located between the second electrode and a third electrode, defining a second electrode pair, the method further comprising:
successively routing an excitation signal through the first electrode pair and the first micropillar array and through the second electrode pair and the second micropillar array during a first measurement interval before the fluid sample is flowing through the microchannel;
successively routing the excitation signal through the first electrode pair and the first micropillar array and through the second electrode pair and the second micropillar array during a second measurement interval after the fluid sample is flowing through the microchannel;
determining a normalized impedance change for the first micropillar array based on electrical impedances measured between the first electrode pair during the first and second measurement intervals; and
determining a normalized impedance change for the second micropillar array based on electrical impedances measured between the second electrode pair during the first and second measurement intervals.

17. The method of claim 12, wherein determining the assessment of deformability and/or microcapillary occlusion further comprises:
estimating a normalized impedance contribution caused by free non-occluding red blood cells present in the microchannel; and
subtracting the normalized impedance contribution from the normalized impedance change determined for the at least one micropillar array to provide a compensated normalized impedance change for the at least one micropillar array, wherein the assessment of deformability and/or microcapillary occlusion of the red blood cells in the fluid sample is determined based on the compensated normalized impedance change.

18. The method of claim 12,
wherein the electrical impedance is measured for each of the micropillar arrays between a respective electrode pair, wherein the electrical impedance measurements for each of the micropillar arrays include at least one measurement before the fluid sample is flowing through the microchannel and at least one measurement after the fluid sample is flowing through the microchannel,
wherein normalized impedance changes are determined for each of the micropillar arrays based on the electrical impedance measurements for each of the respective micropillar arrays before and after the fluid sample is flowing through the microchannel, and
wherein the assessment of deformability and/or microcapillary occlusion of the red blood cells in the fluid sample is determined based on the normalized impedance changes determined for each of the respective micropillar arrays.

19. The method of claim 18, wherein the assessment of deformability and/or microcapillary occlusion is determined as an occlusion index (OI) for the red blood cells in the fluid sample, and the OI is calculated according to the following equation:
$$OI = \frac{( {Z}_{1} - {Z}_{j} ) \cdot {N}_{1} + ( {Z}_{2} - {Z}_{j} ) \cdot {N}_{2} + … + ( {Z}_{m} - {Z}_{j} ) \cdot {N}_{m}}{{N}_{1} + {N}_{2} … + {N}_{m}}$$
$$where :$$
$${Z}_{n} = \frac{{Z}_{n , RBC}}{{Z}_{n , baseline}}$$
Zₙ represents the normalized impedance change determined for an n^{th} micropillar array, where n and j are positive integers from 1 to m, and m+1 represents a total number of the micropillar arrays in the microchannel,
Nₙ represents a total number of openings in the n^{th} micropillar array,
Z_{n,baseline} represents a baseline impedance of the n^{th} micropillar array measured before the fluid sample enters the microchannel, and
Z_{n,RBC} represents the impedance of the n^{th} micropillar array measured after the fluid sample is flowing through each of the plurality of micropillar arrays of the microchannel.

20. The method of claim 12, wherein the fluid sample includes a therapeutic agent to modulate red blood cell adhesion and/or deformability.

21. A method of measuring efficacy of a therapeutic agent in modulating red blood cell adhesion and/or deformability, the method comprising:
perfusing a fluid sample including red blood cells through at least one microchannel of a microfluidic device, wherein the at least one microchannel includes a plurality of micropillar arrays arranged in a direction of fluid flow through the at least one microchannel, and each of the plurality of micropillar arrays is located between a respective pair of electrodes;
measuring an electrical impedance of at least one micropillar array in the at least one microchannel before and after perfusion of the fluid sample through the at least one microchannel, wherein the perfusion of the fluid sample and the measuring of the electrical impedance are performed wash-free;
determining a normalized impedance change for the at least one micropillar array based on the electrical impedance measured before and after the fluid sample is being perfused through the microchannel; and
determining the measure of efficacy of the therapeutic agent based on a comparison of the normalized impedance change for the at least one micropillar array to a control.

22. The method of claim 21, wherein the therapeutic agent is added to the fluid sample that is perfused through the at least one microchannel, and/or
wherein the fluid sample is obtained from a subject having the therapeutic agent administered in vivo.

23. The method of claim 21, further comprising:
adjusting the normalized impedance change for the at least one micropillar array to compensate for a normalized impedance contribution caused by free non-occluding red blood cells present in the microchannel; and
calculating an occlusion index (OI) based on the adjusted normalized impedance change for the at least one micropillar array, wherein the OI is representative of an assessment of deformability and/or microcapillary occlusion of the red blood cells in the fluid sample responsive to the therapeutic agent that has been added, and the measure of efficacy is determined based on a comparison of the OI to a control index, wherein the control index is determined for a fluid sample having a different amount of the therapeutic agent or without the therapeutic agent.
